(19) United States
(12) Patent Application Publication
(10) Pub. No.: US 2013/0331869 A1
(43) Pub. Date: Dec. 12, 2013

(54) Conductive polymeric composites of polycaprolactone fumarate and polypyrrole for nerve regeneration

(71) Applicants: M. Brett Runge, Pine Island, MN (US); Mahrokh Dadsetan, Rochester, MN (US); Michael J. Yaszemski, Rochester, MN (US)

(72) Inventors: M. Brett Runge, Pine Island, MN (US); Mahrokh Dadsetan, Rochester, MN (US); Michael J. Yaszemski, Rochester, MN (US)

(21) Appl. No.: 12/925,182

(22) Filed: Oct. 15, 2010

Related U.S. Application Data
(60) Provisional application No. 61/279,165, filed on Oct. 16, 2009.

(51) Int. Cl. A61F 2/04; A61N 1/05

(52) U.S. Cl. CPC A61F 2/04; A61N 1/0551
USPC 606/152; 607/118; 424/78.32

(57) ABSTRACT

A novel electrically conductive polymer composite composed of polycaprolactone fumarate-polypyrrole (PCLF-PPy) for applications in nerve regeneration is disclosed. The synthesis and characterization of PCLF-PPy and in vitro studies showing PCLF-PPy supports both PC12 cell and Dorsal Root Ganglia neurite extension. PCLF-PPy composite materials were synthesized by polymerizing pyrrole in pre-formed scaffolds of PCLF resulting in an interpenetrating network of PCLF-PPy. PCLF-PPy composite materials possess electrical conductivity up to 6 mS cm⁻¹ with compositions ranging from 5-13.5 percent polypyrrole of the bulk material. Surface topographies of PCLF-PPy materials show microstructures with a RMS roughness of 1195 nm and nanostructures with RMS roughness of 8 nm. PCLF-PPy derivatives were synthesized with anionic dopants to determine effects on electrical conductivity and to optimize the chemical composition for biocompatibility. In vitro studies using PC12 show PCLF-PPy composite materials induce a higher cellular viability and increased neurite extension compared to PCLF. PCLF-PPy composites doped with either naphthalene sulfonic acid or dodecyl benzene sulfonic acid are determined to be the optimal materials for electrical stimulation. In vitro studies showed significant increases in percentage of neurite bearing cells, number of neurites per cell and neurite length in the presence of ES compared to no ES. Additionally, extending neurites were observed to align in the direction of the applied current. Electrically conductive PCLF-PPy scaffolds possess material properties necessary for application as nerve conduits. Additionally, the capability to significantly enhance and direct neurite extension by passing electrical current through PCLF-PPy scaffolds renders them even more promising as future therapeutic treatments for severe nerve injuries.

### CROSS REFERENCE TO RELATED APPLICATION

This nonprovisional patent application claims the benefit of the prior-filed provisional patent application having the provisional application No. 61/279,165 filed on Oct. 16, 2009.

### STATEMENT REGARDING FEDERALLY SPONSORED RESEARCH AND DEVELOPMENT

This invention was made with government support under AR045871 and EB003060 contracts awarded by the National Institutes of Health and under award by the Armed Forces Institute of Regenerative Medicine. The government has certain rights in the invention.

### BACKGROUND OF THE INVENTION

Traumatic injuries resulting in neurological damage to either the central or peripheral nervous system occur frequently. Spinal cord injuries (SCI) affect over 250,000 individuals in the U.S. with 12,000 new cases occurring every year [See Ackery, A.; Tator, C.; Krassioukov, A. A global perspective on spinal cord injury epidemiology. J. Neurotrauma 2004; 21:1355-1370.]. Peripheral nerve injuries (PNI) are more common, with estimates as high as 5 percent of all patients admitted to level 1 trauma [See Taylor, C. A.; Braza, D.; Rice, J. B.; Dillingham, T. The Incidence of Peripheral Nerve Injury Extremity Trauma. Am. J. Phys. Med. Rehabil. 2008; 87:381-385.]. The frequency and disability associated with PNI injury necessitates the need for therapies to restore the loss of function.

The current clinical standard for the treatment of PNI with segmental nerve loss is the use of nerve autografts; which removes a piece of non critical nerve from a secondary site on the body to replace the missing nerve section. This technique has significant drawbacks including donor site morbidity, insufficient donor nerve length, mismatch of diameter between donor nerve and recipient site, misaligned endoneurial tubes, and mismatched regenerating axons. These drawbacks associated with autografts motivate the search for alternate treatment options.

Synthetic materials have great potential for applications as nerve guidance conduits because they can be fabricated with various dimensions, degradation rates, chemical compositions, mechanical properties, micro-architectures, and external geometries [See Ruiter, G. C. d.; Onyeneho, I. A.; Liang, E. T.; Moore, M. J.; Knight, A. M.; Malessy, M. J. A.; Spinner, R. J.; Lu, L.; Currier, B. L.; Yaszemski, M. J.; Windebank, A. J. Methods for in vitro characterization of multichannel nerve tubes. J. Biomed Mater Res 2007; 84:643-651.; Ruiter, G. C. W. d.; Malessy, M. J. A.; Alaid, A. O.; Spinner, R. J.; Engelstad, J. K.; Sorenson, E. J.; Kaufman, K. R.; Dyck, P. J.; Windebank, A. J. Misdirection of regenerating motor axons after nerve injury and repair in the rat sciatic nerve model. Exp. Neurol. 2008; 211:339-350.; Ruiter, G. C. d.; Spinner, R. J.; Malessy, M. J. A.; Moore, M. J.; Sorenson, E. J.; Currier, B. L.; Yaszemski, M. J.; Windebank, A. J. Accuracy of motor axon regeneration across autograft, single-lumen, and multichannel poly(lactic-co-glycolic acid) nerve tubes. Neurosurgery 2008; 63:144-155.; Moore, M. J.; Friedman, J. A.; Lewellyn, E. B.; Mantilla, S. M.; Krych, A. J.; Ameenuddin, S.; Knight, A. M.; Lu, L.; Currier, B. L.; Spinner, R. J.; Marsh, R. W.; Windebank, A. J.; Yaszemski, M. J. Multiple-channel scaffolds to promote spinal cord axon regeneration. Biomaterials 2006; 27:419-429.; Wang, S.; Yaszemski, M. J.; Knight, A. M.; Gruetzmacher, J. A.; Windebank, A. J.; Lu, L. Photo-crosslinked poly(Îμ-caprolactone fumarate) networks for guided peripheral nerve regeneration: Material properties and preliminary biological evaluations. Acta Biomaterialia 2009.; Wang, S.; Lu, L.; Gruetzmacher, J. A.; Currier, B. L.; Yaszemski, M. J. Synthesis and characterizations of biodegradable and crosslinkable poly(e-caprolactone fumarate), poly(ethylene glycol fumarate), and their amphiphilic copolymer. Biomaterials 2006; 27:832-841.].

In addition, therapeutic drugs can be loaded into the scaffolds for controlled release over days or weeks, and cellular therapies, such as stem cells [See Kemp, S. W. P.; Walsh, S. K.; Midha, R. Growth factor and stem cell enhanced conduits in peripheral nerve regeneration and repair. Neurological Research 2008; 30:1030-1038.; Cui, G.-X.; Li, Y.-Z.; Yue, S.-W. Advances in stem cell transplantation for spinal cord injury. Journal of Clinical Rehabilitative Tissue Engineering Research 2008; 12:9335-9338.], adipose derived stromal cells [See Sago, K.; Tamahara, S.; Tomihari, M.; Matsuki, N.; Asahara, Y.; Takei, A.; Bonkobara, M.; Washizu, T.; Ono, K. In vitro differentiation of canine celiac adipose tissue-derived stromal cells into neuronal cells. Journal of Veterinary Medical Science 2008; 70:353-357.], or Schwann cells can be cultured on the scaffolds before implantation [See Tabesh, H.; Amoabediny, G.; Nik, N. S.; Heydari, M.; Yosefifard, M.; Siadat, S. O. R.; Mottaghy, K. The role of biodegradable engineered scaffolds seeded with Schwann cells for spinal cord regeneration. Neurochemistry International 2009; 54:73-83.; Arino, H.; Brandt, J.; Dahlin, L. B. Implantation of Schwann cells in rat tendon autografts as a model for peripheral nerve repair: Long term effects on functional recovery. Scandinavian Journal of Plastic and Reconstructive Surgery and Hand Surgery 2008; 42:281-285.].

Regeneration of damaged nerves faces another obstacle in addition to the above mentioned challenges. As time passes and nerves extend from the proximal to the distal stump regenerating axons and the target organs or muscle increasingly lose their regenerative capacity [See Ashley, Z.; Sutherland, H.; Russold, M. F.; Lanmuller, H.; Mayr, W.; Jarvis, J. C.; Salmons, S. Therapeutic stimulation of denervated muscles: The influence of pattern. Muscle and Nerve 2008; 38:875-886.; Vivo, M.; Puigdemasa, A.; Casals, L.; Asensio, E.; Udina, E.; Navarro, X. Immediate electrical stimulation enhances regeneration and reinnervation and modulates spinal plastic changes after sciatic nerve injury and repair. Experimental Neurology 2008; 211:180-193.; Song, J. W.; Yang, L. J.; Russell, S. M. Peripheral Nerve: What's New in Basic Science Laboratories. Neurosurgery Clinics of North America 2009; 20:121-131.]. Therefore, increasing the rate of nerve regeneration through stimulation may be a critical step to realizing full functional recovery after segmental nerve loss.

Electrical stimulation as a therapeutic treatment for nerve regeneration is gaining increasing interest because of the increasing number of reports showing electrical stimulation increases neurite and axon extension in vitro and nerve regeneration in vivo. Electrical stimulation by either direct exposure to electrical current (AC or DC) or via an electrical field has been shown to have effects on stem cell differentiation [See Kam, N. W. S.; Jan, E.; Kotov, N. A. Electrical stimulation of neural stem cells mediated by humanized carbon nanotube composite made with extracellular matrix protein. Nano Letters 2009; 9:273-278.; Li, L.; El-Hayek, Y. H.; Liu, B.; Chen, Y.; Gomez, E.; Wu, X.; Ning, K.; Li, L.; Chang, N.; Zhang, L.; Wang, Z.; Hu, X.; Wan, Q. Direct-current electrical field guides neuronal stem/progenitor cell migration. Stem Cells 2008; 26:2193-2200.], neurite extension [See Kotwal, A.; Schmidt, C. E. Electrical stimulation alters protein adsorption and nerve cell interactions with electrically conducting biomaterials. Biomaterials 2001; 22:1055-1064.; Schmidt, C. E.; Shastri, V. R.; Vacanti, J. P.; Langer, R. Stimulation of neurite outgrowth using an electrically conductive polymer. Proc. Natl. Acad. Sci. USA 1997; 94:8948-8953.], and influence directionality of growing axons [See Yao, L.; Shanley, L.; Mccaig, C.; Zhao, M. Small applied electric fields guide migration of hippocampal neurons. Journal of Cellular Physiology 2008; 216:527-5351.

Techniques to incorporate electrically conductive materials into biomaterials have included attachment of metal electrodes to proximal and distal nerve stumps [See Ahlborn, P.; Schachner, M.; Irintchev, A. One hour electrical stimulation accelerates functional recovery after femoral nerve repair. Experimental Neurology 2007; 208:137-144.; Geremia, N. M.; Gordon, T.; Brushart, T. M.; Al-Majed, A. A.; Verge, V. M. K. Electrical stimulation promotes sensory neuron regeneration and growth-associated gene expression. Experimental Neurology 2007; 205:347-359.], scaffolds coated with gold nanoparticles [See Park, J. S.; Park, K.; Moon, H. T.; Woo, D. G.; Yang, H. N.; Park, K.-H. Electrical pulsed stimulation of surfaces homogeneously coated with gold nanoparticles to induce neurite outgrowth of PC12 cells. Langmuir 2009; 25:451-457.], and electrically conductive polymers such as polypyrrole [See Shustak, G.; Gadzinowski, M.; Slomkowski, S.; Domb, A. J.; Mandler, D. A novel electrochemically synthesized biodegradable thin film of polypyrrole-polyethyleneglycol-polylactic acid nanoparticles. New J. Chem. 2007; 31:163-168.; Wang, X.; Gu, X.; Yuan, C.; Chen, S.; Zhang, P.; Zhang, T.; Yao, J.; Chen, F.; Chen, G. Evaluation of biocompatibility of polypyrrole in vitro and in vivo. J. Biomed. Mater. Res. 2003; 68:411-422.] or polyaniline [See Huang, L.; Hu, J.; Lang, L.; Wang, X.; Zhang, P.; Jing, X.; Wang, X.; Chen, X.; Lelkes, P. 1.; MacDiarmid, A. G.; Wei, Y. Synthesis and characterization of electroactive and biodegradable ABA block copolymer of polylactide and aniline pentamer. Biomaterials 2007; 28:1741-1751.; Huang, L.; Zhuang, X.; Hu, J.; Lang, L.; Zhang, P.; Wang, Y.; Chen, X.; Wei, Y.; Jing, X. Synthesis of biodegradable and electroactive multiblock polylactide and aniline pentamer copolymer for tissue engineering applications. Biomacromolecules 2008; 9:850-858.].

Schmidt et al. was one of the first researchers to demonstrate that using the conductive polymer polypyrrole and applying an electrical current through the material has a positive effect on neurite extension from PC12 cells [See Schmidt, C. E.; Shastri, V. R.; Vacanti, J. P.; Langer, R. Stimulation of neurite outgrowth using an electrically conductive polymer. Proc. Natl. Acad. Sci. USA 1997; 94:8948-8953.]. Since then numerous groups have thoroughly investigated many aspects of polypyrrole including; in vitro and in vivo biocompatibility, stability, conductivity, incorporation of the cell adhesive polypeptide RGD, and more [See Gomez, N.; Schmidt, C. E. Nerve growth factor-immobilized polypyrrole: bioactive electrically conducting polymer for enhanced neurite extension. J. Biomed. Mater. Res. 2007; 81A:135-149.; Lee, J.-W.; Serna, F.; Nickels, J.; Schmidt, C. E. Carboxylic acid-functionalized conductive polypyrrole as a bioactive platform for cell adhesion. Biomacromolecules 2006; 7:1692-1695.; Lee, J.-W.; Serna, F.; Schmidt, C. E. Carboxy-endcapped conductive polypyrrole:biomimetic conducting polymer for cell scaffolds and electrodes. Langmuir 2006; 22:9816-9819.; Mao, C.; Zhu, A.; Wu, Q.; Chen, X.; Kim, J.; Shen, J. New biocompatible polypyrrole-based films with good blood compatibility and high electrical conductivity. Colloids Surf, B 2008; 67:41-45.; Wang, X.; Gu, X.; Yuan, C.; Chen, S.; Zhang, P.; Zhang, T.; Yao, J.; Chen, F.; Chen, G. Evaluation of biocompatibility of polypyrrole in vitro and in vivo. J. Biomed. Mater. Res. 2003; 68:411-422.].

However, most of this work focuses on thin films of polypyrrole. Although polypyrrole could be very useful for tissue engineering applications, materials composed solely of polypyrrole are not acceptable as biomaterials. PPy has very low solubility in most solvents that make it difficult to process into complex three-dimensional structures and poor mechanical properties that make the materials brittle and weak.

Different approaches have been attempted overcome these limitations and incorporate electrically conductive polymers into biomaterials. Some examples include blending polypyrrole with poly(lactic-co-glycolic acid) [See Shi, G.; Zhang, Z.; Rouabhia, M. The regulation of cell functions electrically using biodegradable polypyrrole-polylactide conductors. Biomaterials 2008; 29:3792-3798.; Shi, G.; Rouabhia, M.; Meng, S.; Zhang, Z. Electrical stimulation enhances viability of human cutaneous fibroblasts on conductive biodegradable substrates. J. Biomed. Mater. Res. 2007; 1026-1036.; Zhang, Z.; Rouabhia, M.; Wang, Z.; Roberge, C.; Shi, G.; Roche, P.; Li, J.; Dao, L. H. Electrically conductive biodegradable polymer composite for nerve regeneration: electricity-stimulated neurite outgrowth and axon regeneration. Artif. Organs 2007; 31:13-22.; Shi, G.; Rouabhia, M.; Wang, Z.; Dao, L. H.; Zhang, Z. A novel electrically conductive and biodegradable composite made of polypyrrole nanoparticles and polylactide. Biomaterials 2004; 25:2477-2488.; Wang, Z.; Roberge, C.; Dao, L. H.; Wan, Y.; Shi, G.; Rouabhia, M.; Guidoin, R.; Zhang, Z. In vivo evaluation of a novel electrically conductive polypyrrole/poly(D,L-lactide) composite and polypyrrole-coated poly(D,L-lactide-co-glycolide) membranes. Journal of Biomedical Materials Research—Part A 2004; 70:28-38.], block copolymers of polylactide and polyaniline [See Huang, L.; Hu, J.; Lang, L.; Wang, X.; Zhang, P.; Jing, X.; Wang, X.; Chen, X.; Lelkes, P. I.; MacDiarmid, A. G.; Wei, Y. Synthesis and characterization of electroactive and biodegradable ABA block copolymer of polylactide and aniline pentamer. Biomaterials 2007; 28:1741-1751.; Huang, L.; Zhuang, X.; Hu, J.; Lang, L.; Zhang, P:; Wang, Y.; Chen, X.; Wei, Y.; Jing, X. Synthesis of biodegradable and electroactive multiblock polylactide and aniline pentamer copolymer for tissue engineering applications. Biomacromolecules 2008; 9:850-858.], nanoparticles composed of polypyrrole-polyethyleneglycol-polylactic acid [See Shustak, G.; Gadzinowski, M.; Slomkowski, S.; Domb, A. J.; Mandler, D. A novel electrochemically synthesized biodegradable thin film of polypyrrole-polyethyleneglycol-polylactic acid nanoparticles. New J. Chem. 2007; 31:163-168.], and templated synthesis of polypyrrole [See Chen, S. J.; Wand, D. Y.; Yuan, C. W.; Wany, X. D.; Zhang, P. Y.; Gu, X. S. Template synthesis of the polypyrrole tube and its bridging in vivo sciatic nerve regeneration. J. Mat. Sci. Lett. 2000; 19:2157-2159.].

### BRIEF SUMMARY OF THE INVENTION

A novel electrically conductive polymer composite composed of polycaprolactone fumarate-polypyrrole (PCLF-PPy) for applications in nerve regeneration is disclosed. The synthesis and characterization of PCLF-PPy and in vitro studies showing PCLF-PPy supports both PC12 cell and Dorsal Root Ganglia neurite extension. PCLF-PPy composite materials were synthesized by polymerizing pyrrole in pre-formed scaffolds of PCLF (Mₙ 7,000 or 18,000 g mol⁻¹) resulting in an interpenetrating network of PCLF-PPy. PCLF-PPy chemical compositions were characterized by ATR-FTIR, XPS, DSC, and TGA. PCLF-PPy composite materials possess electrical conductivity up to 6 mS cm⁻¹ with compositions ranging from 5-13.5 percent polypyrrole of the bulk material. Surface topographies of PCLF-PPy materials were characterized by AFM and SEM show microstructures with a root mean squared (RMS) roughness of 1195 nm and nanostructures with RMS roughness of 8 nm. PCLF-PPy derivatives were synthesized with five different anionic dopants, naphthalene sulfonic acid, dodecyl benzene sulfonic acid, dioctyl sulfosuccinate, iodide, and lysine, to determine effects on electrical conductivity and to optimize the chemical composition for biocompatibility. In vitro studies using PC12 show PCLF-PPy composite materials induce a higher cellular viability and increased neurite extension compared to PCLF. PCLF-PPy composites doped with either naphthalene sulfonic acid or dodecyl benzene sulfonic acid are determined to be the optimal materials for future electrical stimulation and in vivo experiments.

### BRIEF DESCRIPTION OF THE DRAWINGS

FIG. 1a. Chemical structure of polycaprolactone fumarate (top). FIG. 1b. Chemical structure of polypyrrole. FIG. 1c. Single lumen and multi-lumen nerve conduits composed of PCLF-PPy.

FIG. 2. Chemical structures of different anions used to dope polypyrrole.

FIG. 3. ATR-FTIR spectra of PCLF and PCLF-PPy composites. Absorbtion band from 1520-1610 cm⁻¹ is C-C stretches from pyrrole, 1020-1050 cm⁻¹ and 1140-1210 are S═O symmetric and asymmetric stretches respectively and 770-940 cm⁻¹ for S—O stretches of the sulfonate anions.

FIG. 4. XPS regional scans of C, O, N, S, and I showing the absence or presence of peaks for elements associated with PCLF, polypyrrole, and the anionic dopants for a) PCLF. Regional scans of N from polypyrrole and S or I from the anionic dopant for b) PCLF₁₈₀₀₀-PPy_{NSA}, c) PCLF₁₈₀₀₀-PPy_{DBSA}, d) PCLF₁₈₀₀₀-PPy_{DOSS}, and e) PCLF18000-PPy₁.

Table 1. Various compositions and conductivities PCLF-PPy composite materials synthesized with different anionic dopants. ^{a}N⁺ percent of total N species.

Table 2. XPS data showing atomic percent scaffold composition for the top 10 nm and the electrical conductivity related to the scaffold compositions. ^{a}N⁺ percent of total N species. ^{b}Percent polypyrrole determined by thermogravimetric analysis.

FIG. 5. DSC of PCLF₇₀₀₀-PPY_{NSA} as a function of percent polypyrrole composition determined by TGA.

FIG. 6. TGA of varying compositions of PCLF₇₀₀₀-PPy_{NSA}

FIG. 7. SEM micrographs showing different surface topographies of PCLF-PPy scaffolds. PCLF₇₀₀₀PPy_{NSA} (left), PCLF₁₈₀₀₀PPy_{NSA} (right).

FIG. 8. AFM micrographs of surface microstructure (left) with root mean squared roughness (RMS) of 1195 nm and nanostructure with an RMS roughness of 8 nm (right)

FIG. 9. MTS assay showing differences in cell viability of PC12 on polystyrene tissue culture plates (TCP), PCLF and PCLF₁₈₀₀₀-PPy composite materials doped with Lysine, iodide, DOSS, NSA, and DBSA. PCLF and PCLF-PPy materials were compared to TCP using a paired t-test. Materials that showed results significantly greater than TCP are ^{#}p<0.05, ^{##}p<0.01, and ^{###}p<0.001. PCLF-PPy treatments were compared to PCLF. PCLF-PPY materials with significantly higher cell numbers than PCLF are denoted as *p<0.05, **p<0.01, and ***p<0.001.

FIG. 10. Confocal microscopy micrographs at 24 h of PC12 cells cultured on different polymeric materials. The different scaffolds are A & B) PCLF₁₈₀₀₀, C & D) PCLF₁₈₀₀₀-PPy_{NSA}, E & F) PCLF₁₈₀₀₀-PPy_{DBsA}, PCLF₁₈₀₀₀-PPy_{Iodide}, H) PCLF₁₈₀₀₀-PPy_{lysine}.

FIG. 11a. Neurite extension from DRG explants on PCLF-PPy materials doped with NSA, DBSA, and DOSS. FIG. 11b. Fluorescence microscopy image showing a DRG explant on PCLF-PPy_{NSA} after 96 hours.

FIG. 12 shows fluorescence microscopy images of PC12 cells at 10× and 40×.

FIG. 13a shows, for neurite bearing cells, the average number of neurites per cell when applying different stimulation regimens was measured.

FIG. 13b shows the distribution of the number of neurites per cell.

FIG. 13c shows a 40× image of one PC12 cell having been subject to 1 h/day of 10 μA 20 Hz ES and bearing multiple neurite extensions.

FIG. 14a shows the median neurite length and the distribution of neurite lengths.

FIG. 14b shows the distribution of neurites measured for lengths of 10 μm ranges.

FIG. 14c shows the relative distribution where 90% of the neurites of PC12 cells cultured in the absence of ES had lengths of 0-20 μm.

FIG. 15a shows the number of neurites with respect to degrees of current deviation.

FIG. 15b shows the percent of neurites with respect to degrees of current deviation.

### DETAILED DESCRIPTION OF THE INVENTION

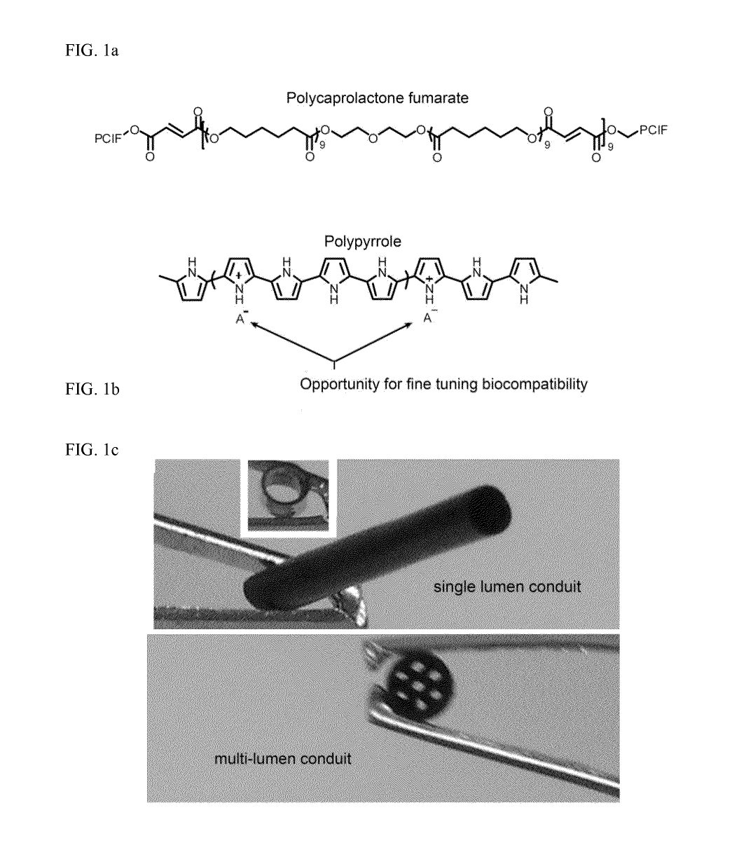

A novel synthetic method to produce composite materials composed of polycaprolactone fumarate (PCLF) and polypyrrole (PPy) is disclosed. PCLF (chemical structure shown in FIG. 1) is a chemical or photo-cross-linkable derivative of polycaprolactone that can be easily processed into complex three-dimensional structures by injection molding. PCLF has been shown to be biocompatible, has good mechanical properties that make it suitable for use in applications for nerve guidance conduits, and have tunable degradation rates [See Wang, S.; Lu, L.; Gruetzmacher, J. A.; Currier, B. L.; Yaszemski, M. J. Synthesis and characterizations of biodegradable and crosslinkable poly(e-caprolactone fumarate), poly(ethylene glycol fumarate), and their amphiphilic copolymer. Biomaterials 2006; 27:832-841.; Jabbari, E.; Wang, S.; Lu, L.; Gruetzmacher, J. A.; Ameenuddin, S.; Hefferan, T. E.; Currier, B. L.; Windebank, A. J.; Yaszemski, M. J. Synthesis, material properties, and biocompatibility of a novel self-cross-linkable poly(caprolactone fumarate) as an injectable tissue engineering scaffold. Biomacromolecules 2005; 6:2503-2511.].

PCLF has previously been shown to direct nerve regeneration in the rat sciatic nerve defect model [See Wang, S.; Yaszemski, M. J.; Knight, A. M.; Gruetzmacher, J. A.; Windebank, A. J.; Lu, L. Photo-crosslinked poly(lμ-caprolactone fumarate) networks for guided peripheral nerve regeneration: Material properties and preliminary biological evaluations. Acta Biomaterialia 2009.], and is currently under in vivo study as nerve guidance conduits in conjunction with therapeutic drugs, Schwann cells, and/or adult adipose-derived stem cells.

However, a major issue with polymeric nerve conduits in general is that regenerating nerve tissue grows through the polymer as a cable and is surrounded by a thick wall of fibrous tissue that does not make any contact with the polymer walls. This significantly restricts the available space for regenerating tissue. Therefore, the development of materials that promote neural cell attachment and decrease fibrous tissue in-growth into the scaffold would represent an attractive improvement to these scaffolds. To increase cellular compatibility and stimulate nerve regeneration PCLF was extended to the electrically conductive PCLF-PPy composite material.

PCLF-PPy polymer composites can be easily fabricated into complex three-dimensional conduits such as single lumen and multi-lumen nerve conduits shown in FIG. 1 and overcome the limitations associated with processing polypyrrole into complex three-dimensional structures.

PCLF-PPy materials maintain the physical properties of the host polymer PCLF. This alleviates the poor mechanical properties associated with using PPy and incorporates the property of electrical conductivity into the scaffold. The synthesis and characterization of this novel electrically conductive composite polymeric material is disclosed. The effect of chemical composition of these polymeric materials on cell viability and neurite extension is shown by comparing different anionic dopants used in the synthesis of PPy.

2. Materials and Methods

2.1 Materials

All chemicals were purchased from Aldrich or Fisher Chemicals and used as received unless noted. Polycaprolactone fumarate (PCLF) was synthesized by previously reported procedures from PCL diol with Mₙ of 2,000 g mol⁻¹. Resulting PCLF had a Mₙ of 7,000 or 18,000 g mol⁻¹ and PDI of 1.96 or 1.82, respectively. Different molecular weight PCLF will be distinguished in this manuscript by the following nomenclature PCLF₇₀₀₀ or PCLF₁₈₀₀₀. The Mₙ and PDI were characterized by GPC using polystyrene standards. The GPC system consists of a Waters 2410 refractive index detector, 515 HPLC pump, and 717 Plus autosampler, and a Styragel HR4E column. THF was used as the eluent at 1 mL/min.

2.2 Synthesis of PCLF-PPy Composite Materials

Phenyl bis(2,4,6-trimethylbenzoyl) phosphine oxide (BAPO) (300 mg, 72 μmol) was dissolved in 3 mL methylene chloride (MeCl₂). Three hundred μL of BAPO solution, PCLF₇₀₀₀ (3.0 g), and MeCl₂ (0.6 mL) were heated and mixed to form a viscous homogenous liquid that was poured into various molds consisting of two glass slides separated by a Teflon spacer to form sheets with thicknesses of 0.5 mm. These molds containing the PCLF mixture where placed in a UV chamber and irradiated for 1 h at λ=315-380 nm to cross-link the PCLF material. The cross-linked PCLF scaffolds were removed from the molds and submerged in MeCl₂ to remove uncross-linked materials and then dried under vacuum. Benzoyl peroxide (1.0 g, 4.1 mmol) was dissolved in MeCl₂ (20 mL). The PCLF scaffold (0.1 g) was submerged in the benzoyl peroxide solution for times ranging from 5 seconds to 5 minutes. The scaffold was removed and then dried under vacuum for 10 minutes to remove residual MeCl₂. Freshly distilled pyrrole (0.56 g, 8.4 mmol) and naphthalene sulfonic acid (NSA) (0.4 g, 1.7 mmol) were dissolved in 20 mL of deionized distilled water and cooled to 0° C. The scaffold was submerged in the aqueous pyrrole solution and stirred overnight. The scaffold was removed, washed with acetone and excessive amounts of water to remove excess dopant and pyrrole, and then swelled in methylene chloride, acetone, and ethanol to remove residual impurities to remove residual impurities before drying under vacuum.

2.3 Characterization of PCLF-PPy Composite Materials

2.3.1 X-ray Photoelectron Spectroscopy (XPS)

The surface elemental composition was characterized on a custom-designed Kratos Axis Ultra X-ray photoelectron spectroscopy system. A complete description of the instrument is given elsewhere [See Baltrusaitis, J.; Usher, C. R.; Grassian, V. Reactions of sulfur dioxide on calcium carbonate single crystal and particle surfaces at the adsorbed water carbonate interface. Phys. Chem. Chem. Phys. 2007; 9:3011-30241. Briefly, the surface analysis chamber is equipped with a monochromated 1486.6 eV aluminum K_{□} source having a 500 mm Rowland circle silicon single crystal monochromator. The typical X-ray gun settings were 15 mA emission current at an accelerating voltage of 15 kV. Low energy electrons were used for charge compensation to neutralize the sample. Survey scans were collected using the following instrument parameters: an energy scan range of 1200 to −5 eV; pass energy of 160 eV; step size of 1 eV; dwell time of 200 ms and an X-ray spot size of 700×300 □m. High resolution spectra were acquired in the region of interest using the following experimental parameters: 20 to 40 eV energy window; pass energy of 20 eV, step size of 0.1 eV and dwell time of 1000 ms. The absolute energy scale was calibrated to the Cu 2p_{2/3} peak binding energy of 932.6 eV using an etched copper plate. A magnetic lens, mounted below the sample, combined with the electrostatic lenses are used to focus the scattered electron beam from the surface. A hemispherical sector analyzer (HSA) was used to analyze the electron kinetic energy, while a delay-line detector measured the electron count.

All spectra were calibrated using the adventitious carbon 1 s peak at 285.0 eV. A Shirley-type background was subtracted from each spectrum to account for inelastically scattered electrons that contribute to the broad background. Commercially available CasaXPS software was used to process the XPS data [See Fairley, N.; 2.3.14., C. V. 1999-2008.]. Transmission corrected relative sensitivity factor (RSF) values from the Kratos library were used for elemental quantification, as implemented into CasaXPS. The components of the peaks contain a Gaussian/Lorentzian product with 30% Lorentzian and 70% Gaussian character. An error of ±0.2 eV is reported for all the peak binding energies.

2.3.2 Attenuated Total Reflectance Fourier Transform Infrared Spectroscopy (ATR-FTIR)

The chemical composition of cross-linked polymeric materials was characterized on a Nicollet 8700 FTIR spectrometer. A Germanium ATR crystal was used at a resolution of 4 cm⁻¹ at 1000 cm⁻¹. Spectra were obtained with a minimum of 64 scans.

2.3.3 Scanning Electron Microscopy (SEM)

Surface topographies were imaged using a Hitachi 4700 field emission spectrometer. PCLF polymer disks were sputter coated with Au/Pd to prevent sample charging during imaging. PCLF-PPy composite materials were imaged uncoated because the materials are electrically conductive and do not require coating to prevent charging. The samples were imaged at an accelerating voltage of 5 kV.

2.3.4 Atomic Force Microscopy (AFM)

Atomic force microscopy images were taken using Asylum Research MFP-3D instrument. NSC15-ALBS cantilevers from MikroMasch with typical resonant frequency of 315 kHz and typical force constant of 40 N/m were used in all experiments. Samples were fixed to a glass slide by the epoxy glue. Images were acquired in air using alternating current (AC) method.

2.3.5 Thermal Analysis

Thermogravimetric analysis (TGA) was performed on a TA Instruments Q500 thermal analyzer. Samples were heated from room temperature to 800° C. at a rate of 1° C. min⁻¹ under flowing nitrogen. Dynamic scanning calorimetry (DSC) was performed on a TA Instruments Q1000 differential scanning calorimeter. Under a nitrogen atmosphere the sample under went a heat-cool-heat cycle to ensure the same thermal history between samples. Samples were heated from room temperature to 100° C., then cooled to −80° C., and then heated to 150° C. at a rate of 5° C. min⁻¹.

2.3.6 Electrical Conductivity

The resistance of polymer films was measured by the 4-point probe methods. The 4-point probe was fabricated based on previous literature reports using the parallel plate model [See Hiremath, R. K.; Rabinal, M. K.; Mulimani, B. G. Simple setup to measure electrical properties of polymeric films. Review of Scientific Instruments 2006; 77.]. Gold electrodes used for the measurement were purchased from Case Western Reserve Electronics Design Center. A electrophoresis DC current source (Hoeffer PS 3000) was used to supply the current. The voltage and current were measured using a Fluke 73 multimeter. The resistance was calculated using the following equation: ρ=4.53 Vwh/IL. 4.53 is the correction factor for samples with h<0.5 L, V is volts measured, I is current measured, w is width of sample, h is sample thickness, and L is sample length between electrodes.

2.4 PC12 Cell Culture Studies

2.4.1 PC12 Cellular Response to PCLF-PPy Composite Materials

Tissue Tek 24 well cell culture plates, medical grade silicon tubing of inner diameter 0.95 cm, DMEM media PCLF-PPy composite materials were fabricated into disks of diameters 1.0 cm as described above, sterilized with 70% ethanol and used as is. Toxicity of residual starting materials leaching from PCLF-PPy scaffolds was evaluated using a non contact method. PC12 cells were seeded in 12 well plates at a density of 20,000 cells cm⁻² for 24 h prior to the addition of the polymeric material contained in trans wells. PC12 cells were cultured in the presence of polymeric materials for 1 day and then the cell numbers was quantified with an MTS assay and the trans wells were transferred to fresh wells containing cells and cultured for another 3, and 7 days.

To investigate PC12 cell response to different polymeric materials 1.0 cm disks were placed in a 24 well plates. The scaffolds were sterilized in 70% aqueous ethanol for 30 minutes and then rinsed with sterile PBS. Medical grade silicon tubing that had been autoclaved was then inserted into the well to limit the surface area of the polymer disk to a diameter of 0.95 cm with a surface area of 0.71 cm². The well was filled with media and incubated for 12 hours to remove any remaining impurities. PC12 cells were plated at a density of 30,000 cells cm⁻². Experiments were performed with NGF (50 ng mL⁻¹) supplemented media. Cell viability was determined using MTS (Promega, Madison, Wis.) assays. 0.5 mL trypsin was added to each well, aspirated, and put in the incubator for 10 min. 0.5 mL media was then added to each well and cells were gently dislodged from the surface with a cell scraper. Media and cells were then transferred to a new well and 0.1 mL of MTS reagent was added to each well then and incubated for 2 h at 37° C. The absorbance was measured at 490 nm on a Molecular Devices spectra max plate reader. Cell morphology was imaged by fluorescence microscopy. PC12 cells on polymer scaffolds were fixed in 2% parafomaldehyde in PBS for 25 min, and then washed with PBS three times. Cells were permeablized in 0.1% Triton 100× for 3 min and then incubated in 10% horse serum in PBS for 1 h. Cells were stained in 1% rhodium phalloidin in 5% horse serum in PBS for 1 h and then washed with PBS three times. Nuclei were stained with DAPI just prior to mounting on a glass cover slip. Samples were imaged on an LSM 510 inverted confocal microscope and imaged at excitation wavelengths of 368 and 488 nm.

2.4.2 Statistics

Experiments were performed with triplicate specimens and results are reported as mean±standard deviation. Single factorial analysis of variance (ANOVA) was performed to determine statistical significance of the data. When a global F-test showed a significant difference at the p<0.05 level a paired t-test was used to determine significant differences between treatments.

3. Results

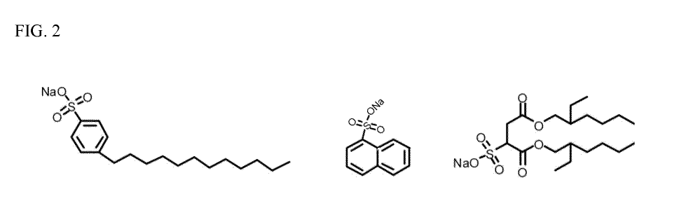

Electrically conductive PCLF-PPy composite materials were synthesized by polymerizing pyrrole in preformed cross-linked PCLF scaffolds. The resulting interpenetrating network (IPN) of PPy and PCLF resulted in scaffolds that appeared to be macroscopically homogenous and were colored black characteristic of PPy. Because subtle differences in chemical composition can have large impacts on scaffold properties PPy was synthesized with five different anionic dopants to investigate the effect different anions have on the electrical conductivity and cell attachment. The sodium salts of three sulfonic acid analogs where chosen (dodecyl benzene sulfonic acid, naphthalene sulfonic acid, and dioctyl sulfosuccinate shown in FIG. 2) in addition to iodide, and lysine. The effect of anionic dopant on both electrical conductivity and cell attachment proved to be an important factor for PCLF-PPy materials.

3.1 Characterization of PCLF-PPy Composite Materials

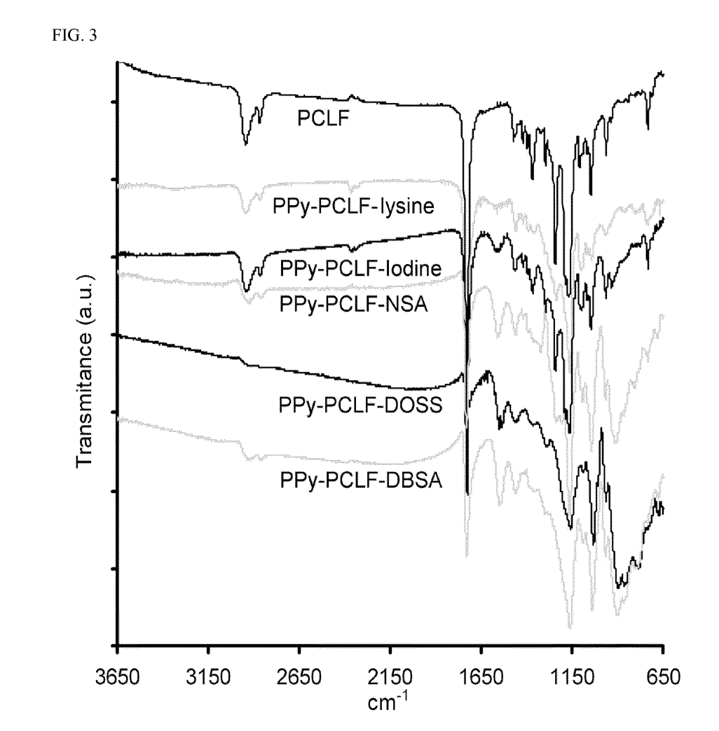

Scaffolds of PCLF-PPy where characterized by ATR-FTIR to confirm the presence of polypyrrole. FIG. 3 shows the ATR-FTIR spectra of PCLF and PCLF-PPy composite materials synthesized with different anionic dopants. The appearance of a strong band from 1520-1610 cm⁻¹ present in all PCLF-PPy spectra and clearly absent in the PCLF spectra is characteristic of skeletal C—C stretches from the pyrrole ring. PCLF-PPy materials doped with sulfonic acid anions also show absorption bands corresponding to the sulfonate group at 1020-1050 cm⁻¹ and 1140-1210 from to S═O symmetric and asymmetric stretches respectively and 770-940 cm⁻¹ for S—O stretch.

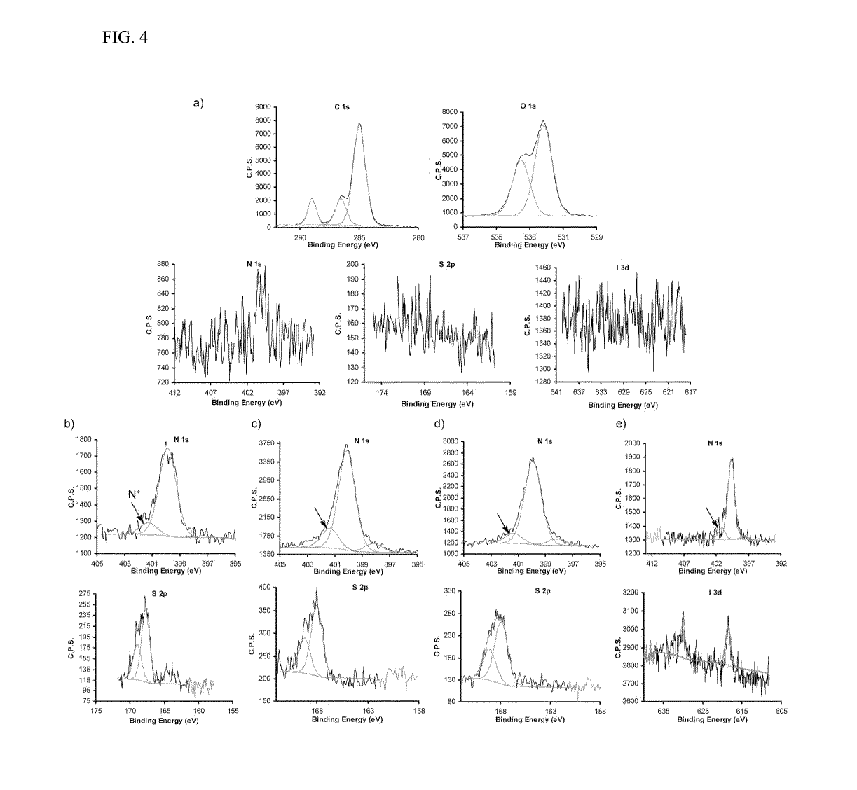

The surface elemental composition of PCLF and PCLF-PPy composite materials were characterized by XPS to verify the presence of polypyrrole and anions on the surface of PCLF-PPy scaffolds. XPS was also used to quantify the amount of polypyrrole incorporated onto the surface and percent of polypyrrole that is doped with anions resulting in the conductive polypyrrole species. XPS spectra of PCLF shown in FIG. 4a shows no detectable amounts of nitrogen, sulfur, or iodide. FIG. 4b shows regional scans for N and S or I for the different PCLF-PPy composites. Table 1 presents the elemental composition quantified by XPS of the numerous scaffolds, the corresponding electrical conductivity, and the bulk composition determined by TGA. All PCLF-PPy composite materials show the presence of N and S with the up to 6 atomic percent nitrogen that corresponds to 30 mol percent polypyrrole incorporated into the top 10 nm of the scaffold surface. The percent of N⁺ of total nitrogen incorporated into the scaffold indicates that polypyrrole is nearly fully doped and corresponds with the atomic percent of S or 1.

The scaffolds electrical conductivity was influenced by the anionic dopant selection. PCLF-PPy_{NSA} and PCLF-PPy_{DBSA} had the highest conductivity of 6 mS cm⁻¹, iodide had 0.1 mS cm⁻¹, and lysine doped PCLF-PPy exhibited no measureable conductivity. Table 1 shows most scaffolds had conductivity near 1 mS cm⁻¹. Scaffolds with varying percents of polypyrrole where fabricated to investigate the effect of compositional on electrical conductivity. PCLF₇₀₀₀-PPy_{NSA} was synthesized with up to 3 atomic percent nitrogen incorporated into the scaffold shown in Table 2. The amount of polypyrrole incorporated into PCLF was controlled by the amount of oxidant occluded with in the PCLF scaffold by varying benzoyl peroxide concentrations and times that PCLF was submerged in these solutions. PCLF-PPy composite materials with at least 3% N have conductivities around 1 mS cm⁻¹. These conductivities should be more than adequate to incorporate the electrical current necessary for future applications.

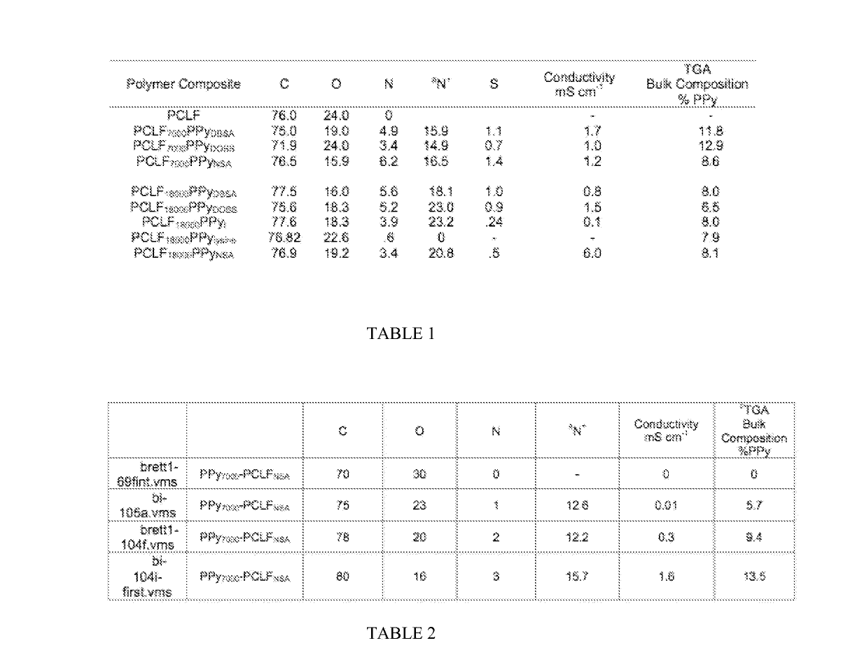

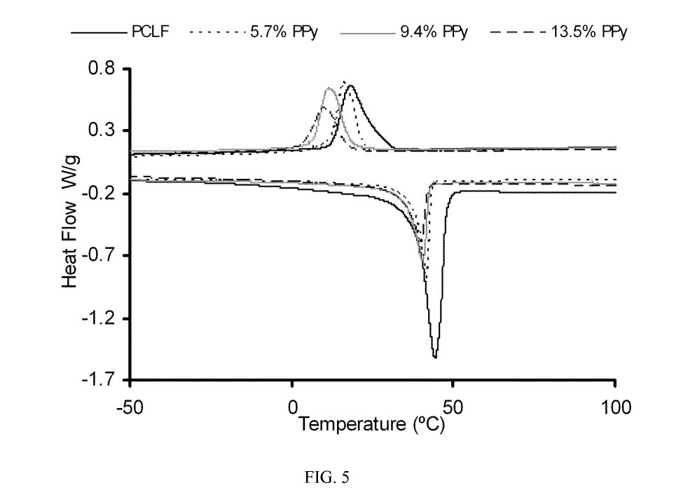

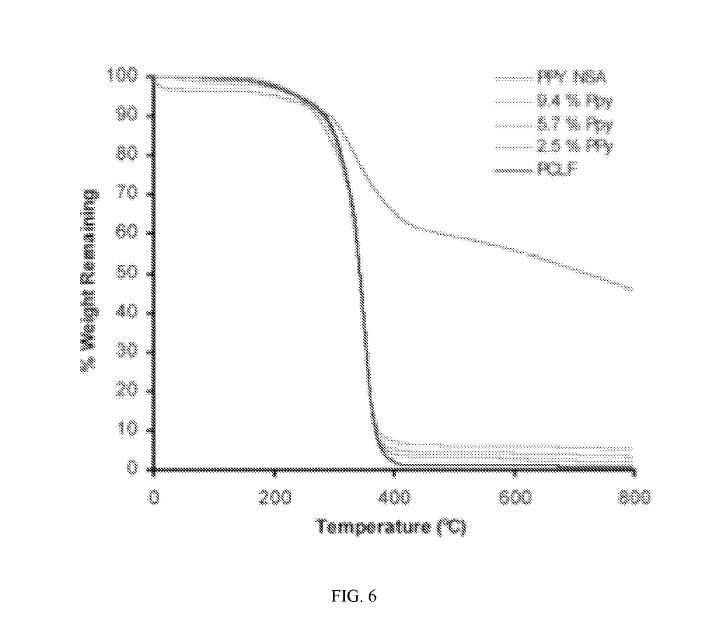

Controlling the percent polypyrrole incorporated into the scaffolds can be used to tune the conductivity and physical properties of PCLF-PPy. The amount of PPy incorporated into each of the composite materials can be controlled by the amount of benzoyl peroxide occluded within the PCLF scaffold during the synthesis of polypyrrole. FIG. 5 shows select TGA runs of PCLF-PPy showing up to 13.5% polypyrrole successfully incorporated into the scaffold bulk material. The percent composition is influenced by the molecular weight of the preformed PCLF scaffolds. Because PCLF₇₀₀₀ has a lower cross-linking density and higher swelling ratio than PCLF₁₈₀₀₀, PCLF₇₀₀₀ is able to incorporate a higher amounts of PPy into the scaffold than PCLF₁₈₀₀₀ with the highest being 13.5% PPy of the bulk composition. The thermal transitions of PCLF and PCLF-PPy composites were investigated by DSC. PCLF is a semi-crystalline material with Tₘ and T_{c} transitions that straddle the 37° C. body temperature. FIG. 5 shows the DSC traces for different PCLF-PPy composites as a function of bulk composition. DSC indicates that PCLF₇₀₀₀ has a Tₘ of 45° C. that lowers to 42, 41, and 40° C. with increasing polypyrrole composition. PCLF also has a T_{c} of 18° C. that decreases to 16, 11, and 9° C. with increases in polypyrrole. Depression of both the Tₘ and T_{c} temperatures translates to a more amorphous material at physiological temperature that results in increased flexibility that is desirable for application in nerve guidance conduits.

3.2 Surface Characterization by SEM and AFM

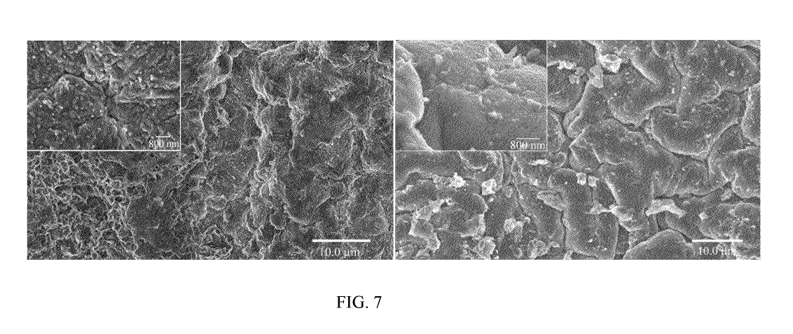

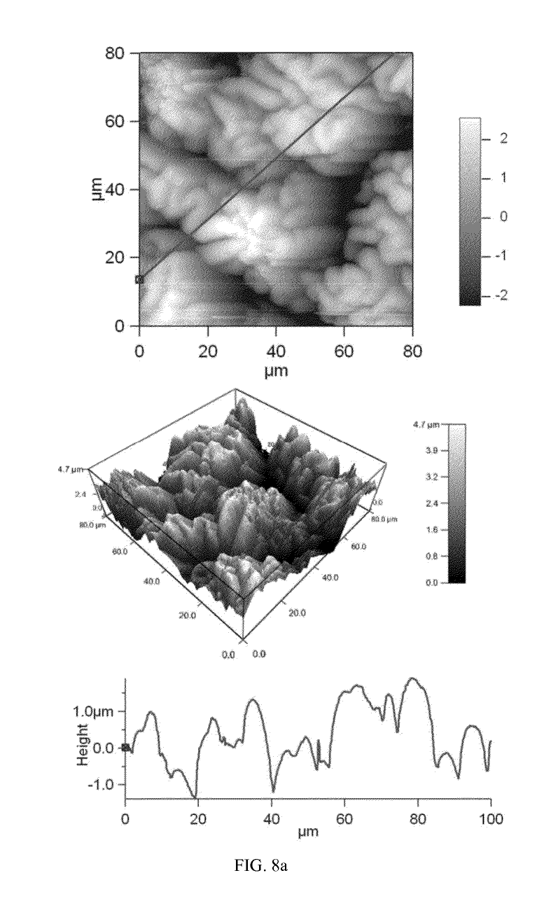

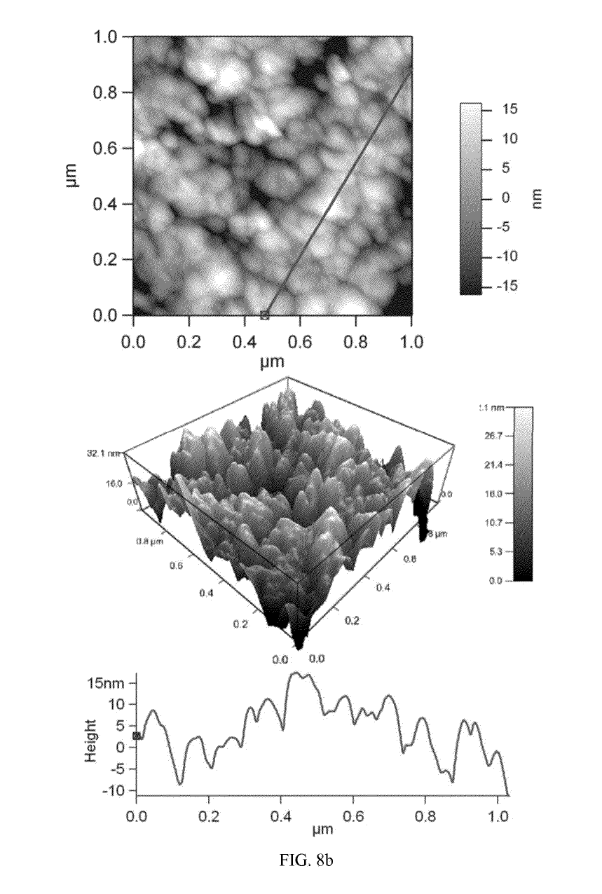

Scaffold surface topography is an important factor influencing cell attachment and can either hinder or promote cell attachment. It is generally known that rougher surfaces promote cell attachment, and it is important to be able to control this scaffold property. FIG. 7 shows scanning electron micrographs of PCLF₇₀₀₀-PPy_{NSA} and PCLF₁₈₀₀₀-PPY_{NSA} polymer composites with varying degrees of surface roughness. The surface roughness is controlled by the sol-gel fraction of the polymer composite. Polymer composites consisting of PCLF₇₀₀₀ with low cross-linking have higher soluble fractions that are swelled out of the composite material with organic solvents resulting in an increased surface roughness. PCLF₁₈₀₀₀ has a cross-linking density roughly three times higher than the PCLF₇₀₀₀ resulting in a decreased sol fraction and smoother surface. PCLF_{7,000}-PPy_{NSA} has a rougher surface (FIG. 7, left), than PCLF_{18,000}-PPy_{NSA} (FIG. 7, right). Similar differences in surface roughness were observed by AFM. The AFM micrographs of PCLF₁₈₀₀₀PPY_{DBSA} are shown in FIG. 8. AFM shows that the PCLF₁₈₀₀₀PPy_{DBSA} has root mean squared roughness (RMS) of 1195 nm over tens of microns, while the RMS is 8 nm over 1 micron. The granular microstructure observed in the FIG. 8 is characteristic of polypyrrole, while the macrostructure is due to the PCLF polymer scaffold. The effect of these differences in surface roughness where investigated for effects on cell attachment.

3.3 In Vitro Evaluation and Comparison of Materials

The PC12 cell line was used for in vitro evaluation of cell attachment, proliferation, and morphology on PCLF-PPy scaffolds as well as toxicity of leaching materials from the scaffolds. Because of toxicity associated with starting materials from the synthesis of polypyrrole, unreacted materials were extensively extracted from PCLF-PPy scaffolds by swelling in methylene chloride followed by acetone prior to cell culture experiments. Initial cytotoxicity evaluations were performed, by plating cells 24 h prior to the addition of PCLF-PPy materials suspended within a transwell. PC 12 cells remain viable for the duration of the experiment, which totaled 11 days. No decrease in cell viability was observed for cells cultured on tissue culture plates in the presence of PCLF or PCLF-PPy scaffolds indicating no toxicity of leaching materials from novel PCLF-PPy scaffolds.

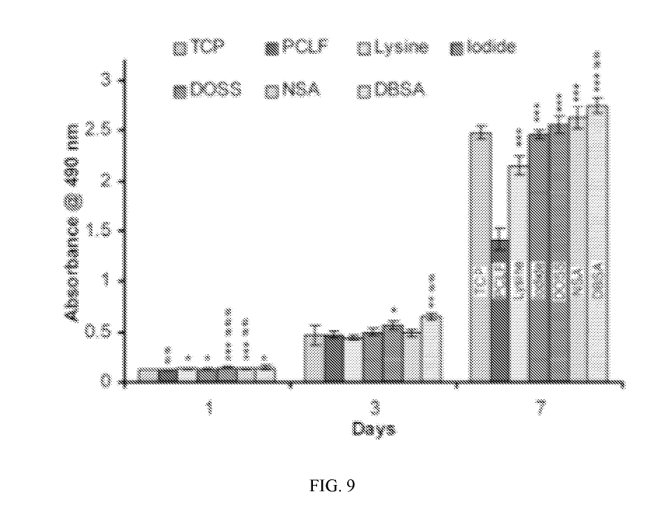

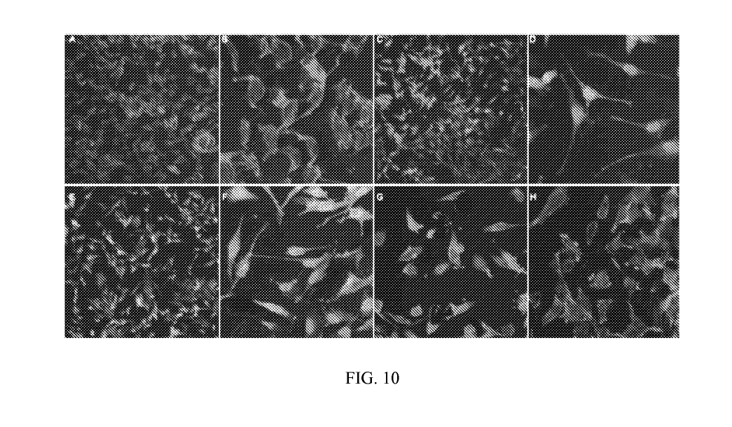

PC12 cells were plated on the surface of PCLF and PCLF-PPY scaffolds at a density of 30,000 cells cm⁻². Cell attachment and proliferation were characterized by MTS assay at days 1, 3, and 7. Polystyrene tissue culture plates were used as a positive control. In order to quantify cell numbers with the MTS reagent, the PC12 cells were trypsinized from the polymer scaffolds and transferred to a new well without the polymeric material. This was necessary because interference due to interactions between the MTS dye or resulting formazan product and the PCLF-PPy scaffolds resulted in an artificially low absorbance values. This interaction was not unexpected as the MTS dye contains a sulfonate functional group and may coordinate to the positively charged polypyrrole. FIG. 10 shows that incorporation of polypyrrole into PCLF scaffolds enhances PC12 cell attachment and proliferation on PCLF-PPy over PCLF scaffolds. Significant differences were observed at day 1 for the initial PC12 cell attachment. All PCLF-PPy showed significant increases in cell attachment (p<0.05). Large significant differences between PCLF and all PCLF-PPy composite materials could be seen at day 7 (FIG. 9) (p<0.001). This indicates that PC12 cells have a greater proliferation rate on the surface of PCLF-PPY scaffolds in comparison to unmodified PCLF. PC12 cells also attached better on PCLF-PPy_{NSA} and PCLF-PPy_{DBSA} composite materials doped than on the tissue culture plates. The effect of differences in surface roughness between PCLF₇₀₀₀PPy and PCLF₁₈₀₀₀PPy on cell attachment were also investigated. Although distinct differences between the surface morphologies PCLF₇₀₀₀PPy and PCLF₁₈₀₀₀PPy materials were observed by both SEM and AFM no significant advantage was observed for cell attachment to either scaffold.

PC12 cell morphology is an important indicator of neuronal differentiation, and can be influenced by subtle chemical cues from the scaffolds. Morphologies of PC12 cells cultured on PCLF or PCLF-PPy scaffolds show distinct differences between the materials. Cell morphologies shown in FIG. 10 were imaged after 24 h by fluorescence microscopy after staining with a rhodium phalloidin stain for f-actin and DAPI stain for nuclei. PC12 cells cultured on PCLF have round morphology with few small neurites. However, PC12 cells cultured on The PCLF₁₈₀₀₀-PPy_{NSA} and PCLF₁₈₀₀₀-PPy_{DBSA} exhibit a typical morphology for differentiating PC12 cells extending their neurites on the surface of scaffolds. The rest of the PCLF-PPy scaffolds show many cells with unfavorable round morphologies and few neurites.

3.4 Dorsal Root Ganglia Explants

Dorsal root ganglia (DRG) where extracted from rat embryos and cultured on PCLF-PPy materials doped with DOSS, NSA, and DBSA. DRG explants include nerve cells with supporting cells such as Schwann and Glial cells that play important roles in neuron extension. FIG. 11a shows that PCLF-PPy materials can support DRG attachment and neurite extension, and that the DRG response is influenced by the dopant used in the composite material. This result matches the effect of dopant on cell morphology seen with PC 12 cells with NSA and DBSA materials performing the best. PCLF-PPy_{NSA} exhibited a mean neurite extension of 690±253 um, PCLF-PPy_{DBSA} had 536±297 um, and PCLF-PPy_{DOSS} shows 17±68 um. DRG explants do not attach to PCLF. FIG. 11b shows the fluorescence microscopy of a DRG explant cultured on PCLF-PPy_{DBSA} after 96 h.

3.5. Electrical Stimulation of PC12 Cells on PCLF-PPy_{NSA} Scaffolds

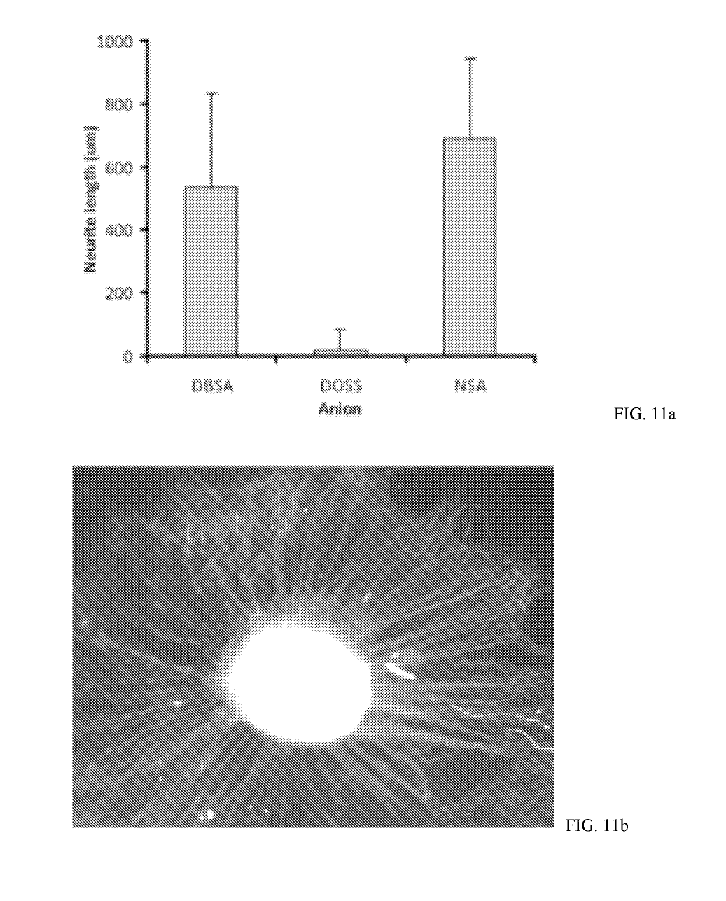

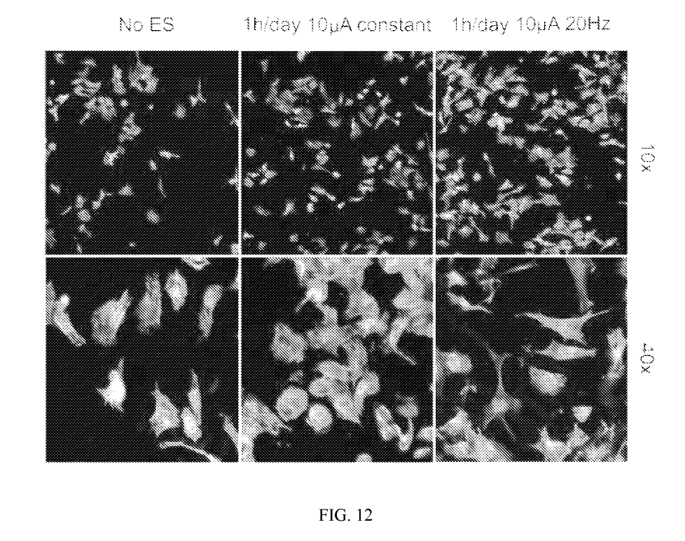

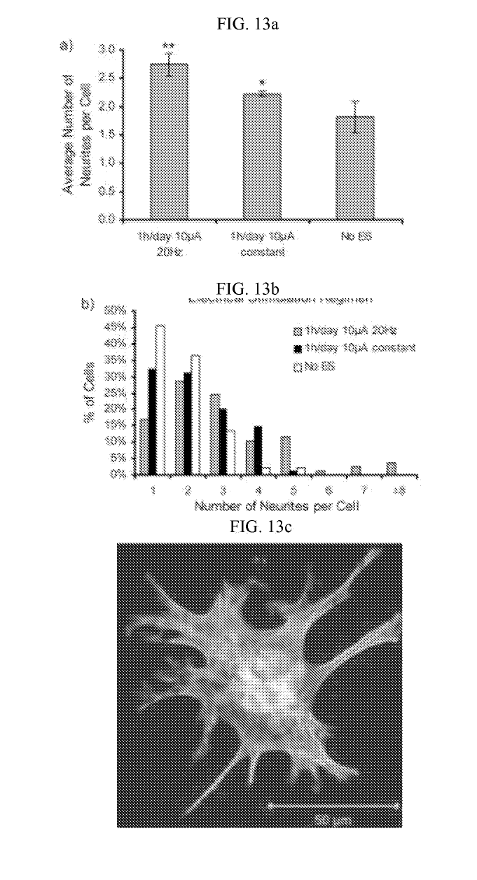

PC12 cells were chosen as the model cell line for the initial studies involving electrical stimulation because they are a commonly used cell for nerve regeneration studies, and there is ample literature precedence to compare the results of electrical stimulation treatments. FIG. 12 shows fluorescence microscopy images of PC12 cells at 10× and 40×. The 10× images show typical images used to analyze neurite extensions through NIH image J software. Distinctly more and longer neurites can be seen when electrical stimulation (ES) was applied than without electrical stimulation. The percentage of PC12 cells bearing neurites was 75.7% for 1 h/day of 10 μA of constant current and 83.0% for 1 h/day of 10 μA of 20 Hz frequency. These results were significantly higher (p<0.01) than the 49.7% observed with no ES. No statistically significant difference was observed between ES treatment regimens. For neurite bearing cells, the average number of neurites per cell when applying different stimulation regimens was measured (FIG. 13a). Compared to cells not exposed to ES, PC12 cells that received ES treatment demonstrated an increase in amount of neurites per cell from 1.8 to 2.7 for stimulation of 20 Hz and 2.2 for constant stimulation (p<0.01). FIG. 13b shows the distribution of the number of neurites per cell. Of the neurite bearing PC12 cells cultured in the absence of ES 46% had 1 neurite, 82% had two or less and 96% had 3 or less neurites. ES stimulation shifted the distribution to higher numbers of cells bearing 2 or more neurites. 32% had one neurite, 31% had two neurites, 20% had three neurites, and 16% of the PC 12 cells exposed to constant stimulation had 4 or more neurites, up from the 5% observed with no ES. PC12 cells cultured in the presence of 20 Hz ES exhibited the most substantial shift in number of neurites per cell. Only 17% of these PC12 cells had one neurite, while 30% had 4 or more neurites. FIG. 13c shows a 40× image of one PC12 cell having been subject to 1 h/day of 10 μA 20 Hz ES and bearing multiple neurite extensions.

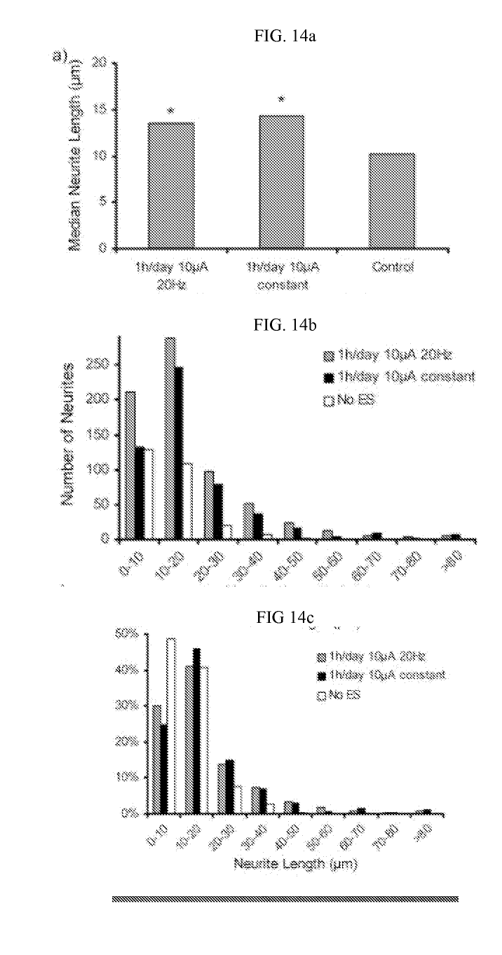

The median neurite length and the distribution of neurite lengths are shown in FIG. 14a. Cells stimulated with ES showed significant increases (p<0.01) in median neurite length from 10.2 μm with no ES to 14.4 μm for constant ES and 13.6 μm for 20 Hz ES (FIG. 14a). No significant difference was observed between the two ES regimens. FIG. 14b,c show the distribution of neurites measured for lengths of 10 μm ranges. FIG. 14b shows that the 20 Hz ES treatments had the highest counts in most length categories of the neurite length distribution, consistent with the results from FIG. 13b. FIG. 14c shows the relative distribution where 90% of the neurites of PC12 cells cultured in the absence of ES had lengths of 0-20 μm. PC12 cells with either ES treatment exhibited 71% of cells having neurites of 0-20 μm, but they were distributed differently with constant and 20 Hz ES having 46% and 41% of neurites ranging from 10-20 μm.

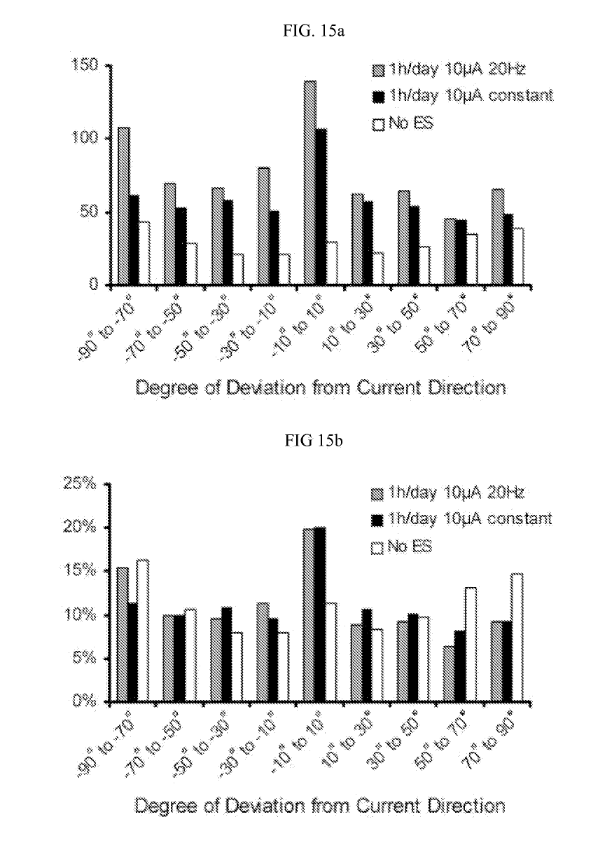

The effect of ES on the direction at which neurites extend was investigated by measuring the angle of the neurites in relation to the direction of the applied current. FIG. 15a displays the distribution of neurite alignment showing a doubling in the number of neurites within a range of ±10° parallel to the current direction for both ES treatment regimens. FIG. 15b shows the percent of neuritis with respect to degrees of current deviation. When no ES was applied this peak was absent, and the angles at which the neurites were extending were evenly distributed. This indicates a preferential alignment of neurites with the direction of the stimulating current if ES is applied on cells.

### DISCUSSION

Polypyrrole has gained increasing interest in biomaterials over the last decade because of the positive effect electrical stimulation has been shown to have on tissue regeneration. However polypyrrole has poor mechanical properties resulting in weak and brittle materials making it unsuitable for applications in peripheral nerve regeneration. Composite materials that incorporate a small amount of polypyrrole with another polymer that has suitable material properties can overcome limitations of polypyrrole. This methodology motivated the development of PCLF-PPy materials. PCLF-PPy was synthesized by polymerizing pyrrole in preformed cross-linked PCLF scaffolds. Cross-linked polymeric materials have advantages over non-cross-linked materials because they swell in organic solvents but do not dissolve. This results in materials that are more robust for post fabrication modification by allowing the occlusion of small molecules within the cross-linked polymer matrix while maintaining the original geometric shape of complex 3-dimensional scaffolds. Benzoyl peroxide, the initiator for polymerization of pyrrole, was occluded within cross-linked PCLF scaffolds by submerging scaffolds in a solution of benzoyl peroxide in methylene chloride. The cross-linked scaffold swells as methylene chloride and benzoyl peroxide diffuse in. Subsequent removal of methylene chloride by evaporation leaves benzoyl peroxide occluded within the PCLF scaffold. PCLF scaffolds containing benzoyl peroxide are then submerged in aqueous solutions of pyrrole. Pyrrole diffuses into the scaffold and is rapidly polymerized resulting in an interpenetrating network (IPN) of PCLF and PPy. This methodology for creating IPNs is robust and should be able to be applied to many different types of cross-linked polymeric materials.

PCLF was synthesized from PCL diol with a Mn of 2000 g mol⁻¹ and fumaryl chloride. Two different molecular weight PCLF polymers were synthesized with Mₙ of 7,000 or 18,000 g mol⁻¹. Two different molecular weight PCLF polymers were synthesized in order to investigate any effect the PCLF molecular weight may have on the material properties and biocompatibility of PCLF-PPy composite materials. The difference between PCLF₇₀₀₀ and PCLF₁₈₀₀₀ is the cross-link density of the resulting polymer matrix. Cross-linking density affects thermal transitions, sol-gel fraction, and swelling ratio of the cross-linked materials. The thermal transitions studied include T_{c} and Tₘ. Cross-linked PCLF has T_{c} and Tₘ that straddle the 37° C. Polymerizing PPy in PCLF scaffolds lowers the both thermal transitions resulting in more amorphous materials and increased flexibility under physiological conditions. The swelling ratio and sol-gel fractions of cross-linked polymer matrices can affect the diffusion of small molecules into the material and the surface topography of the final PCLF-PPy scaffolds. PCLF₇₀₀₀ and PCLF₁₈₀₀₀ did not have substantially different electrically conductivity, both had conductivities on the order of 1 mS cm⁻¹. Surface topographies between the two different PCLF materials were visually different with PCLF₇₀₀₀ having a rougher surface, but no difference in cellular response was observed. Because the subtle differences between the two PCLF molecular weight materials did not have an effect on cell response the PCLF₁₈₀₀₀ is preferred because of its increased flexibility when dry, and increased strength during processing due to a lower degree of swelling.

The synthesis of PPy requires selection of an anionic dopant that stabilizes the positive charges formed along the conjugated pi system that is responsible for the conductivity of PPy. This anionic dopant allows for some variation in chemical composition and material properties. This initial study used five different anions. Three sulfonic acid analogs and iodide were chosen because they have been previously used to dope PPy and resulting materials had high electrical conductivity. The fifth, lysine, was chosen because it is zwitter ion and an amino acid. The five dopants were investigated to fine tune the electrical and biological properties of the scaffolds. The sulfonic acid derivatives (NSA, DBSA, and DOSS) had the highest conductivity on the order of 1-10 mS cm⁻¹, PCLF₁₈₀₀₀-PPy₁ measured 0.1 mS cm⁻¹, and PCLF₁₈₀₀₀-PPy_{lysine} had no measurable conductivity. Differences in conductivity maybe attributed to the ability of the different anionic dopants to diffuse into the PCLF polymer matrix during polymerization of pyrrole. If diffusion of the anionic dopant into PCLF is limited, PPy will stay in the reduced non-conductive form.

PC12 is a cell line derived from a pheochromocytoma of the rat adrenal medulla. These cells stop dividing and differentiate into the neurons when treated with nerve growth factor. In this study, we used PC12 cells as a model system for neuronal differentiation, and investigation of cells response to the novel PCLF-PPy composite materials. PCLF is currently being investigated in vitro for peripheral nerve regeneration [See Wang, S.; Yaszemski, M. J.; Knight, A. M.; Gruetzmacher, J. A.; Windebank, A. J.; Lu, L. Photo-crosslinked poly(Îμ-caprolactone fumarate) networks for guided peripheral nerve regeneration: Material properties and preliminary biological evaluations. Acta Biomaterialia 2009.]. The goal of these experiments was at minimum to find an electrically conductive PCLF-PPy composition that performs well as the PCLF, but also possessed the electrically conductive properties. Therefore five anionic dopants were investigated for PC12 cell response. PC12 cells cultured on PCLF-PPy materials attached equally well on PCLF. At 7 days PC12 cells have the lowest cell number on PCLF materials. This result may be due to the poor cell attachment on PCLF and removal of cells during media changes. PC12 cell morphologies cultured on the different materials is shown in FIG. 11. The PC12 cells are stained with a rhodium phalloidin dye that stains f-actin. The f-actin is a critical protein for attachment and cytoskeleton organization of the cells. They can be seen as the red strands with in the cell bodies after staining. PC12 cells cultured on PCLF₁₈₀₀₀ (FIG. 11 A,B) show very little F-actin staining compared to PC12 cells on PCLF-PPy materials. Lack of f-actin expression can be an indicator of poor attachment [See Dadsetan, M.; Jones, J. A.; Hiltner, A.; Anderson, J. M. Surface chemistry mediates adhesive structure, cytoskeletal organization, and fusion of macrophages. Journal of Biomedical Materials Research—Part A 2004; 71:439-448.]. Fluorescence microscopy images show obvious differences between the materials. Cells cultured on PCLF₁₈₀₀₀-PPy_{NSA} and PCLF₁₈₀₀₀PPy_{DBSA} show a typical differentiating morphology in the presence of NGF. No round cell morphologies were observed as is seen with the other materials. These cells exhibit extended cell bodies with multiple long straight neurites extending from the cell.

DRG explants are an excellent model that utilizes neurons with all the supporting cells investigate a treatments ability to support neurite outgrowth. In this study DRG explants were used to confirm the observations of neurite extension with PC12 cells. PCLF-PPy_{DBSA}, PCLF-PPy_{NSA}, and PCLF-PPy_{DOSS} where chosen because they had superior properties for electrical conductivity and PC12 cell response. These experiments show that PCLF-PPy_{NSA} and PCLF-PPy_{DBSA} is able to support DRG attachment and neurite extension over the PCLF-PPY_{DOSS} composite material, and PCLF alone does not support attachment of DRG.

The main interest in PCLF-PPy scaffolds is their electrical conductivity and being able to pass electrical current through them to stimulate growing nerve cells. PCLF-PPy networks doped with NSA and DBSA anions are preferred because they demonstrate the best material properties and most extensive cell attachment. The Rₛ for PCLF-PPy_{NSA} is as low as 2 kΩ thus exhibiting a similar conductivity as for other PPy composite materials. Additionally, the scaffolds are electrically stable when different ES treatment regimens are applied, especially if NSA was the utilized dopant. This combined with the fact that compared to PCLF-PPy_{DBSA} PCLF-PPy_{NSA} samples are able to adsorb a higher amount of NGF to their surface makes this the preferred type of scaffold for cell culture experiments. PC12 cells were electrically stimulated with 10 μA of current for 1 h per day. The current was either constant direct current or direct current with a frequency of 20 Hz. The amount of current, 10 μA, was chosen because it renders a surface current density of 7.2 μA/cm₂ which induced the most favorable cell response from a previous study. The frequency of 20 Hz was chosen because 20 Hz is considered an effective frequency for stimulating nerve regeneration in the rat model and in humans. Using ES with a frequency of 20 Hz instead of constant current is preferred because it resembles average firing frequencies of motor neurons. Even though the electrical stability of the samples was tested with three different time regimens, the cells were only subject to the 1 h/day ES treatment because it is a commonly used regimen when trying to stimulate neuronal regeneration in vivo.

Image analysis of PC12 stimulated with a frequency of 20 Hz revealed an impressive 67% increase in the percentage of neurite bearing cells, dramatically higher than the 5% recently achieved in another study with similar PPy-PCL scaffolds. Using PPy coated PLGA nanofibers to apply ES treatments on PC12 cells, an even higher relative increase of 92% was reported, however, in that case no increase in numbers of neurites per cell was found. In comparison, when treatments of 1 h/day 10 μA 20 Hz ES were applied on cells seeded on PCLFPPy_{NSA} samples the average number of neurites per cell increased by 52%. Considering the length of the extending neurites when electrical stimulation regimens are applied relative increases of 30%, 48.8%, 50%, and 90.5% are reported in the literature. The 33.0% increase achieved when 10 μA 20 Hz ES were applied on PCLF-PPy_{NSA} scaffolds might therefore seem low at first sight, however it has to be taken into consideration that the increase in cells bearing multiple neurites leads to a shift of the median neurite length to lower values, because the neurites grow longer, but at the same time many new short ones appear. This explanation is corroborated by the observation that constant ES, which had a lower relative increase of the average number of neurites per cell showed a higher relative increase of 41.0% in the median neurite length. The exact mechanism of action of ES on neurons is not fully understood, but might involve increased adsorption of the extracellular matrix protein fibronectin to the scaffold surface, changes in membrane potential, and the vectored accumulation of surface glycoproteins. According to our results, pulsed ES treatments of 20 Hz led to a significant increase of the number of neurites per cell when compared to constant stimulation. Considering the widely accepted use of pulsed ES treatments of 20 Hz to aid nerve regeneration in vivo and the finding that PC12 cells show increased viability when stimulated with pulsed rather than constant treatments, the use of pulsed ES is preferred for enhancing neurite extension on electrically conductive scaffolds for improved results.

Since outgrowing axons need to find the desired trajectory towards and into the distal endoneurial tubes, micropatterned surfaces, microchannels or aligned nanofibers have been investigated for their potential to guide outgrowing nerve axons, rendering promising results. When analyzing the orientation of neurite extension, alignment with the current direction was found. Combined with the fact that electrical fields are able to influence the direction of neurite extension in general, this finding indicates an additional method to guide axon outgrowth within nerve conduits by applying electrical current.

### CONCLUSIONS

Electrically conductive composite materials were synthesized by polymerizing PPy in preformed cross-linked scaffolds of PCLF resulting in interpenetrating networks of PCLF-PPy. This fabrication technique removes the challenges associated with using PPy in biomaterial applications such as poor mechanical properties, processing difficulties, and non-biodegradability. PCLF-PPy materials were synthesized with five different anionic dopants to determine the optimal composition for both the electrical and biological properties. PCLF-PPy_{NSA} and PCLF-PPy_{DBSA} materials exhibited conductivity up to 6 mS cm⁻¹. Surface analysis by XPS indicates the scaffolds contain up to 30 mol percent of polypyrrole in the surface 10 nm. The TGA shows that the bulk material incorporates up to 13.5 percent polypyrrole by weight showing that the majority of the scaffold is biodegradable by hydrolysis. Cellular studies show PC12 cells cultured on PCLF-PPy materials perform better than when cultured on PCLF. However not all PCLF-PPy materials are equal, PCLF-PPy_{NSA} and PCLF-PPy_{DBSA} consistently show better cell morphologies indicated by elongated cell bodies and long neurites extending straight out from the cell in addition to higher cell numbers than other PCLF-PPy composite materials.

PCLF-PPy is a promising material for incorporating electrically conductive materials into tissue engineering. This methodology for producing composite of polypyrrole into preformed cross-linked materials is robust and will be extended to hydrogels and collagen based materials currently being investigated for nerve regeneration applications.

While particular embodiments of the present invention have been described, it will be obvious to those skilled in the art that changes and modifications can be made without departing from the spirit and scope of the teachings and embodiments of this invention. One skilled in the art will appreciate that such teachings are provided in the way of example only, and are not intended to limit the scope of the invention.

1. An electrically conductive composite material comprising polycaprolactone fumarate (PCLF) and polypyrrole (PPy).

2. The material of claim 1 wherein the material increases cellular compatibility and stimulates nerve regeneration.

3. The material of claim 1 wherein the material promotes neural cell attachment.

4. The material of claim 1 wherein the material increases neurite extension.

5. The material of claim 1 wherein the material decreases fibrous tissue in-growth into a scaffold.

6. The material of claim 1 wherein the material is biocompatible.

7. The material of claim 1 wherein the material is used in applications for a nerve guidance conduit.

8. The material of claim 1 wherein the material has tunable degradation rates.

9. The material of claim 1 wherein the material is used for direct nerve regeneration.

10. The material of claim 1 wherein the material is fabricated into a three-dimensional conduit.

11. The material of claim 10 wherein the material is fabricated into a single lumen conduit.

12. The material of claim 10 wherein the material is fabricated into a multi-lumen nerve conduit.

13. The material of claim 1 wherein the material is used in applications for nerve guidance conduits in conjunction with therapeutic drugs, Schwann cells, and/or adult adipose-derived stem cells.

14. The material of claim 1 wherein the PPy is synthesized with anionic dopants.

15. The material of claim 14 wherein the dopants are selected from a group consisting of:
iodide, lysine, dodecyl benzene sulfonic acid, naphthalene sulfonic acid, and dioctyl sulfosuccinate.

16. The material of claim 1 wherein the material has electrical conductivity up to 6 mS cm⁻¹ with compositions ranging from 5-13.5 percent polypyrrole of the bulk material.

17. The material of claim 1 wherein the amount of polypyrrole incorporated into PCLF is controlled by the amount of oxidant occluded within the PCLF scaffold by varying benzoyl peroxide concentrations and times that PCLF is submerged therein.

18. The material of claim 1 wherein the amount of polypyrrole incorporated into PCLF is controlled by the amount of benzoyl peroxide occluded within the PCLF scaffold during the synthesis of polypyrrole.

19. A scaffold comprising an electrically conductive PCLF-PPy composite material.

20. The scaffold of claim 19 wherein the material is synthesized by polymerizing polypyrrole in a preformed cross-linked PCLF scaffold.

21. The scaffold of claim 19 wherein a resulting interpenetrating network (IPN) of PPy and PCLF results in a macroscopically homogenous scaffold.

22. The scaffold of claim 19 wherein the material has a presence of N and S with the up to 6 atomic percent nitrogen corresponding to 30 mol percent polypyrrole incorporated into the top 10 nm of the scaffold surface.

23. A method of manufacture of an electrically conductive material comprising synthesizing a PCLF-PPy composite material by polymerizing polypyrrole in a preformed cross-linked PCLF scaffold.

24. The method of claim 23 further comprising occluding small molecules within the cross-linked scaffold while maintaining an original geometric shape of a complex 3-dimensional scaffold.

25. The method of claim 23 further comprising hydrogels and collagen-based materials for nerve regeneration applications.

26. The method of claim 23 further comprising controlling the percent polypyrrole incorporated into the scaffold to tune the conductivity and physical properties of PCLF-PPy.

27. The method of claim 23 wherein the PPy is synthesized with anionic dopants.

28. The method of claim 27 wherein the dopants are selected from a group consisting of: iodide, lysine, dodecyl benzene sulfonic acid, naphthalene sulfonic acid, and dioctyl sulfosuccinate.

29. The method of claim 23 wherein the material has electrical conductivity up to 6 mS cm⁻¹ with compositions ranging from 5-13.5 percent polypyrrole of the bulk material.

30. The method of claim 23 wherein the amount of polypyrrole incorporated into PCLF is controlled by the amount of oxidant occluded within the PCLF scaffold by varying benzoyl peroxide concentrations and times that PCLF is submerged therein.

31. The method of claim 23 wherein the amount of polypyrrole incorporated into PCLF is controlled by the amount of benzoyl peroxide occluded within the PCLF scaffold during the synthesis of polypyrrole.

32. The method of claim 23 further comprising controlling scaffold surface topography by controlling by the sol-gel fraction of the material.

33. A method of stimulating nerve cell growth comprising:
a. synthesizing an electrically conductive material comprising a PCLF-PPy composite material by polymerizing polypyrrole in a preformed cross-linked PCLF nerve conduit scaffold;
b. applying an electrical current to the material in the nerve conduit scaffold.

34. The method of claim 33 wherein further comprising aligning the current with the direction of the nerve conduit scaffold.

35. The method of claim 33 wherein the current has a frequency of 20 Hz.

36. The method of claim 33 wherein the amount of current is 10 μA.
